(19) United States
(12) Patent Application Publication
(10) Pub. No.: US 2011/0224597 A1
(43) Pub. Date: Sep. 15, 2011

(54) IMPLANT FOR INSERTING INTO THE SCHLEMM'S CANAL OF AN EYE

(75) Inventors: Robert Christopher Stegmann, Pretoria (ZA); Matthias Christian Grieshaber, Binningen (CH)

(73) Assignee: GRIESHABAR OPHTHALMIC RESARCH FOUNDATION C/O PRICEWATERHOUSE COOPERS AG NEUMARKET 4/KorNHAUSST, GALLEN (CH)

(21) Appl. No.: 13/131,400

(22) PCT Filed: Dec. 10, 2010
(86) PCT No.: PCT/EP09/66814

§ 371 Date: May 26, 2011

(30) Foreign Application Priority Data
Dec. 22, 2008 (CH) 2005/08
Nov. 12, 2009 (CH) 174/09

(51) Int. Cl. A61M 5/00

(52) U.S. Cl. 604/8

(57) ABSTRACT

An implant for inserting into an exposed Schlemm's canal is proposed. The implant insertable into the lumen of Schlemm's canal includes an elongated tube which includes an axially extending continuous connecting part having several openings or recesses distanced from each other by web shaped ring members and connecting to the interior of the tube with and with arc shaped surfaces, such that in inserted condition the webs are bearing in supporting manner against the inner wall of the lumen of Schlemm's canal. The openings/recesses form a direct and permanently open connection between the trabecular tissue and the small channels of the channel system for natural trabecular drainage of the aqueous humor.

The invention refers to an implant for inserting into Schlemm's canal of an eye, that has been exposed by incising a section of the sclera to form a scleral flap, the implant consisting of a longitudinal flexible tube that can be inserted up to at least one fourth in the circumference direction into the lumen of the circular Schlemm's canal and having a plurality of openings distanced from each other.

### OPTHALMOLOGICAL BACKGROUND

In a healthy eye, the drainage of the circulating aqueous humor (humor aquosus) from the posterior chamber to the anterior chamber takes place in the chamber angle (angulus iridocornealis), via the trabecular meshwork into Schlemm's canal, and from there carried is away into the blood circulation via the episceral vein system. In pathological conditions of the eye, in particular, when resistances are incurred based on a blocked Schlemm's canal, perhaps due to conglutination, a continuous drainage of the aqueous humor, produced and constantly renewable by the epithelium of the ciliary body, is no longer sufficiently warranted. As a result, the pressure in the interior of the eye (IOP) can rise in such a manner that the blood circulation of the optical nerve and thus, the function thereof is diminished, whereby this dysfunction, defined as the eye disease known as glaucoma or “green star”, can lead to the total blindness of the afflicted eye.

### PRIOR ART

For improvement and maintenance of the anatomical drainage of the aqueous humor, elongated tubes are known from the publications (EP 0898 947 A2 and EP 1 125 568 A2) that are provided with openings, or elongated tube shaped networks, or similarly formed support elements, which are insertable and releasable when inserted into the Schlemm's canal that has been exposed by incision of a section of the sclera forming a scleral flap, and injected with highly viscous medium. By means of the elongated support elements, the anatomically natural drainage of the circulating and constantly renewable aqueous humor traveling from the anterior chamber, via the trabecular tissue, into the lumen of the Schlemm's canal, and from there via the episcleral vein system into the blood circulation, is supposed to be realized.

Furthermore, from the publication (US 2004/0210181 A1), a T-shaped implant attachable to a plate and insertable through an incision in the sclera is known, which comprises a proximal piece of tubing operatively insertable directly into the anterior chamber, or insertable through the trabecular tissue and comprising two distal tubes, oriented opposite each other, for insertion into the exposed Schlemm's canal. The implant which is configured for drainage, in the case of a pathological blockage of the trabecular tissue, the constantly renewable aqueous humor is guided through an artificially created pathway from the proximal tube, inserted into the anterior chamber, via the distal tube, flowing directly into the Schlemm's canal and from there, via the episcleral vein system flows into the blood circulation of the eye, in order to avoid an elevated interior pressure (IOP).

From the two U.S. Publications (US 2005/0192527 A1 and 2007/0088432 A1), further implants are known for the treatment of glaucoma that are either configured with thermal or mechanical shape memory effect and can be formed into an approximate T-shape or, without the shape memory, substantially into a T-shape. These implants are insertable, each with a proximal tube, either directly or operatively, through the trabecular tissue into the anterior chamber and with two oppositely oriented distal tubes arranged at the proximal end, into Schlemm's canal, such that the continuously renewing aqueous humor is likewise carried through an artificial pathway from the anterior chamber directly into Schlemm's canal, and from there carried via the episceral vein system into the blood circulation of the eye.

To reduce the interior pressure, publication (WO 2008/002377 A1), furthermore discloses an implant which comprises several support bodies that are provided with a circular shaped surface and are arranged on an elongated thread or similar, and in a row, and by means of a correspondingly shaped device to be inserted and placed through a scleral incision, for example, fully circumferential, or as a single circular shaped flexible segment part, into the lumen of the exposed circular Schlemm's canal.

In the generally known canaloplasty method, there is the further possibility of a circumferential dilation, whereby the Schlemm's canal is circularly expanded by means of an inserted flexible microcatheter and at the same time, or subsequently, injected by means of a so-called microscrew, with a high molecular viscoelastic agent. Subsequently, the microcatheter is retracted with suitable means, for example, with a surgical thread, and the circular Schlemm's canal is stretched toward the anterior chamber to thus realize an expansion of the trabecular tissue as well as an increased flow with favorable transtrabecular drainage of the aqueous humor.

### ILLUSTRATION OF THE INVENTION

The object of the invention is to provide an implant, insertable into the Schlemm's canal by means of which a circulation of the aqueous humor regulating the interior pressure of the eye across the entire circular lumen of the Schlemm's canal can be realized and to improve and permanently maintain the transtrabecular drainage of the aqueous humor via the episcleral vein system into the blood circulation of the eye.

The implant of the present invention according to the preamble of claim 1, is characterized in that the elongated tube includes: two connecting parts that are oriented in axial direction and circumferentially arranged diametrically opposite each other, several web-shaped ring members arranged at a distance from each other in axial direction, as well as openings between each of the ring members that connect to the interior of the tube and arranged circumferentially at the tube between the first and the second connecting part and opposite each other, each provided with an opening angle oriented in circumferential direction.

Embodiments and limitations as well as details of the invention become evident from the following description in connection with the drawing and each of the patent claims.

The implant according to the invention when inserted into the lumen of Schlemm's canal has the advantage that Schlemm's canal is thereby permanently opened and stabilized. The implant extends at least along one half in circumferential direction, preferably along the entire circumference of Schlemm's canal, such that it can be kept open over the entire circumference, and the anatomical natural transtrabecular drainage of the aqueous humor established via the episcleral vein system into the blood circulation and thereby regulation of the interior pressure of the eye realized.

To optimize the transtrabecular drainage of the aqueous humor, it is possible to insert and place the implant according to the present invention after a circumferential dilation of Schlemm's canal into the expanded lumen of Schlemm's canal.

### DESCRIPTION OF THE DRAWINGS

The drawings accompanying the drawings show:

FIG. 1 a longitudinal section of an eye shown schematically and enlarged;

FIG. 2 a front view in schematic illustration with a parabolic incision in the sclera and an open scleral flap;

FIG. 3 a portion of the eye shown in an enlarged view and according to plane A-A as in FIG. 2, with the partially exposed Schlemm's canal;

FIG. 4 a section of the eye in an enlarged view with an injection probe inserted into Schlemm's canal;

FIG. 5 a section of the exposed Schlemm's canal according to FIG. 4 in an enlarged view, with an inserted and placed implant in the lumen;

FIG. 6 a front view of a first embodiment of the implant made from a circular ring- or oval shaped tube;

FIG. 7 a cross section of the circular ring-shaped implant shown in an enlarged view and according to plane B-B as in FIG. 6;

FIG. 8 a variant of the implant shown in an enlarged view with the oval shaped cross section according to plane B-B in FIG. 6;

FIG. 9 a front view of a second embodiment of the implant made from a circular ring shaped and oval shaped elongated tube;

FIG. 10 the implant shown in an enlarged view with the circular ring shaped cross section according to plane C-C in FIG. 9;

FIG. 11 a variant of the implant with the circular ring shaped cross section according to FIG. 10;

FIG. 12 a variant of the implant shown in an enlarged view, with the circular oval shaped cross section according to plane C-C in FIG. 9; and

FIG. 17-18 further variants of the implant of FIG. 6.

### DESCRIPTION OF THE FIGURES

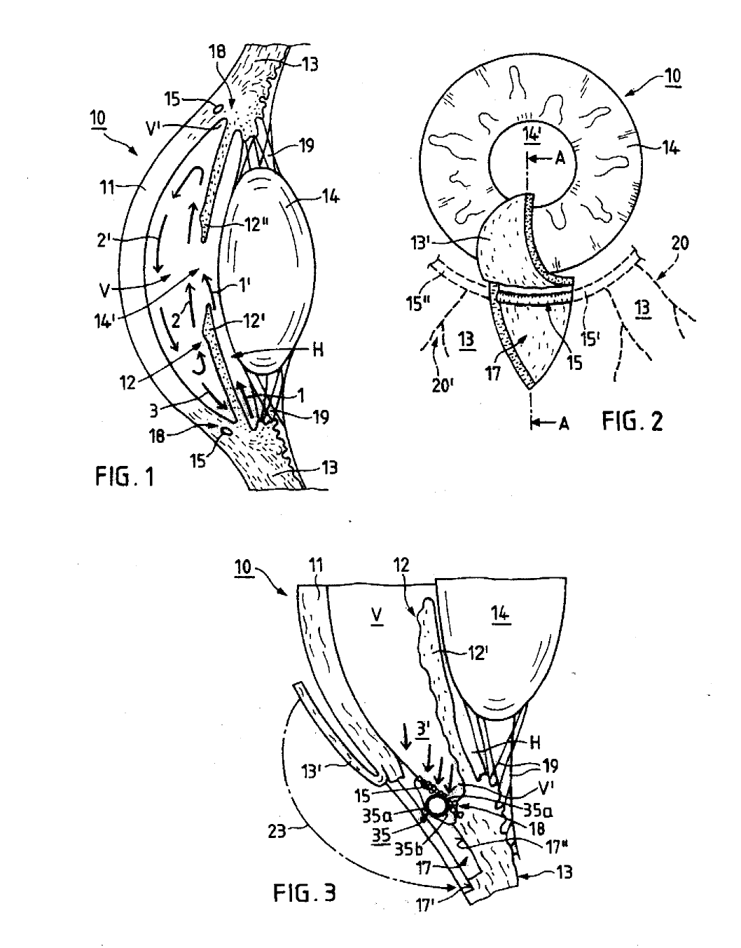

It is pointed out here that in FIGS. 1 to 5, only a section of the eye is illustrated for better understanding of the problem in connection with glaucoma surgery. Furthermore, in each of the figures, the same elements are provided with the same numerals throughout the following description.

FIG. 1 shows the anterior section of an eye 10, which is already known from the illustration in publication EP 0 898 947, where the cornea 10 is shown, the iris 12 with two regions 12′ and 12″, the sclera 13, the lens 14 with the pupil 14′, the zonula fibers 19, the circular Schlemm's canal 15 (sinus venosus sclerae), as well as the trabecular tissue 18 (trabeculum corneosclerale) in front of Schlemm's' canal 15.

As schematically illustrated in FIG. 1, in a healthy eye, the drainage of the circulating and constantly renewable aqueous humor (humor aquosus) according to the drawn arrows 1,1 and 2,2, from the posterior chamber H to the anterior chamber V, takes place in the area of the chamber angle (angulus iridocornealis) according to direction of arrow 3, via the trabecular tissue 18 into the lumen of the circular Schlemm's canal 15, and from there reaches again the blood circulation via the episcleral vein system, not shown in FIG. 1.

As afore-described, in pathological conditions of the eye, a continuous drainage of the aqueous humor, which is produced by the epithelial tissue of the ciliary body and constantly renewed, can no longer be realized. Schlemm's canal 15 can close up in such a way that the drainage of the aqueous humor is obstructed or, to a large part prevented, so that the interior pressure in the eye rises to such a degree that the blood circulation of the optical nerves is diminished in such a way, that as a result, the so limited function leads to blindness in the eye.

FIG. 2 shows a schematic illustration of the eye 10 also known from the publication EP 0 898 947, in a front view with the lens 14 with pupil 14′, a section of the sclera 13, a section of Schlemm's canal 15 as well as a section of the canal system 20, 20′ (aqueous humor channel system), which connects to Schlemm's canal. Schlemm's canal 15 which is partially shown as a schematic illustration extends circumferentially via an angle of 360° and extends circularly around the lens 14.

By means of a microsurgical procedure, a lamellar incision is made in sclera 13 and after separation of a scleral portion, not shown here in detail, the outer section 13′ of the scleral flap lifted open and held there by means not shown here in detail for any further surgical procedures. The lamellar incision in the area of the exposed Schlemm's canal 15 forms a scleral bed 17 which, after further procedures, for example, after the insertion and placement of the elongated implant, according to the direction of arrow 23 (FIG. 3) will again be closed up by lowering the section 13′ (scleral flap).

In a further variant of the microsurgical procedure, it is possible that the trabecular tissue 18 (FIG. 3) which is located anterior to Schlemm's canal 15, is being at least partially circularly opened for the insertion and placement of the implant, by means of a cutting instrument not shown here which has been inserted into the anterior chamber V.

FIG. 3 shows, in an enlarged view of the section of the eye 10 according to line A-A of FIG. 2, with the cornea 11, the first area 12′ of the iris 12, the sclera 13 with the scleral flap 13′, the lens 14, the zonula fibers 19, the posterior chamber H and the anterior chamber V, with chamber angle V′, the trabecular tissue 18 and Schlemm's canal 15 with the implant 35 disposed therein. The Schlemm's canal 15 extending, as shown schematically and enlarged in FIG. 3, essentially along the trabecular tissue 18, and the profile of its cross section shaped like an elongated oval which, starting from one end in the area of the chamber angle V′, in the direction of the other end, essentially has a tapered shape. Furthermore, FIG. 3 shows the scleral bed 17 exposed by the incision and interior surface 17″ with the support surface 17′ for scleral flap 13′.

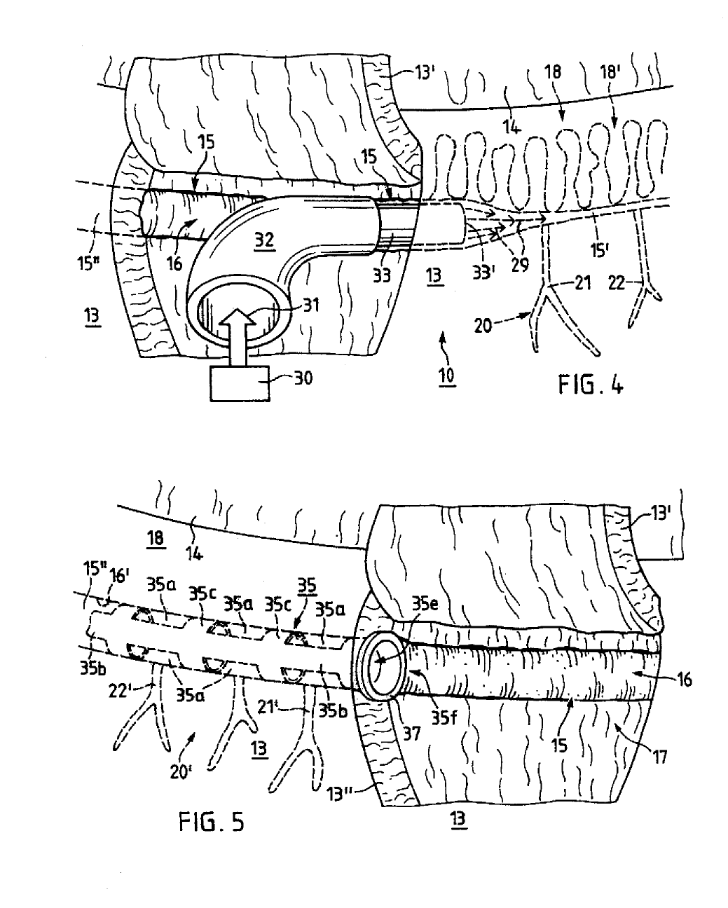

FIG. 4 shows in known manner how a tube shaped probe 33, disposed at a connector piece 32, is inserted into the lumen 16 of the exposed Schlemm's canal 15. The connector piece 32 is connected via a supply line, not shown here, to a schematically illustrated injection device 30. With the aid of the injection device 30, the tube shaped probe 33 with at least one exit opening 33′ at the distal end, for example, a hydrophilic liquid 29 will be injected into Schlemm's canal 15 according to the direction of arrow 31, and as a result, hydraulically expands in circumferential direction a section 15′ of Schlemm's canal 15.

Furthermore, and in known manner Schlemm's canal 15 can be dilated by means of a probe configured mirror image like and inserted into a section 15″ of Schlemm's canal 15 located opposite the section 15′ that has already been treated, to carry out the hydraulic dilation in circular direction. Also shown in FIG. 4 is the trabecular tissue 18 (trabecular meshwork), located anterior to Schlemm's canal 15, with schematically illustrated tissue webs 18′ as well as the canal system 20 with small channels 21 and 22.

During the afore-described dilation of Schlemm's canal 15, the openings in the wall (not shown here) are at the same time optionally charged with a hydrophilic liquid 29, such that the hydrophilic liquid, which is clinging to the walls of the openings in the form of a film, prevent a local tissue connection to thus realize drainage of the aqueous humor. Instead of the hydrophilic liquid, a suitably biocompatible gaseous medium, or a mixture of hydrophilic liquids and the gaseous medium can be utilized for the dilation of Schlemm's canal.

As schematically illustrated in FIG. 5, following the hydraulic or pneumatic expansion, an implant 35 is inserted into the lumen 16 of the circular Schlemm's canal 15 in order to optimize a permanent permeability for, and circulation of the aqueous humor. The implant 35 consists of an elongated flexible tube 36 and is preferably made from biocompatible flexible material and inserted into the lumen 16 of Schlemm's canal by suitable means, not shown here in detail, for example, a probe (inserting instrument) or similar.

FIG. 5 shows furthermore, a section of the implant 35 inserted into Schlemm's canal 15 which is detachably disposed at the proximal end (nearest to the inserting instrument) of the probe (inserting instrument). At the other, distal end, (farthest from the inserting instrument), the implant 35 has an opening 35f with an abutment collar 37 which bears against the interior side 13″ of sclera 13. The implant 35 inserted into the lumen 16 of Schlemm's canal extends from one of the interiors sides 13″ of the exposed Schlemm's canal, not shown here in detail, up to at least one forth, one half, three quarter or preferably, the entire circumference, up to the opposite interior side (FIG. 2). In a variant, not shown here, it is possible to insert an essentially semicircular shaped implant 35 into the exposed Schlemm's canal, each from the one side and from the opposite side of the lamellar incision. With implant 35, the lumen 16 of the circular Schlemm's canal 15 is supported and permanently kept open.

FIG. 5 further illustrates the sclera bed 17 which is formed through the lamellar incision between the two interior sides 13″ opposite each other forming a subscleral or a collection reservoir for the aqueous humor, when the scleral flap is lowered and supported by the parabolic support surface 17′ and sewn together with the sclera 13. The scleral bed 17 is connected with the interior space 35c via the two openings 35f opposite each other (only one opening 35f is shown) of the implant 35.

Further shown in FIG. 5 is a section of the implant 35 inserted into Schlemm's canal 15 adjacent the interior wall 16′ of the lumen 16 and supporting same by means of the ring members 35c that are distanced from each other. The openings or recesses 35a between each of the ring members 35c each form, as illustrated in FIG. 5, a direct and permanently opened connection between the trabecular tissue 18 and each of the small channels 21′ and 22′ of canal system 20′, so that the natural transtrabecular drainage of the aqueous humor from the anterior chamber V, via the trabecular tissue 18 into the circular Schlemm's canal 15, or into the interior 35e of implant 35 and from there via the episcleral vein system into the blood circulation, is realized.

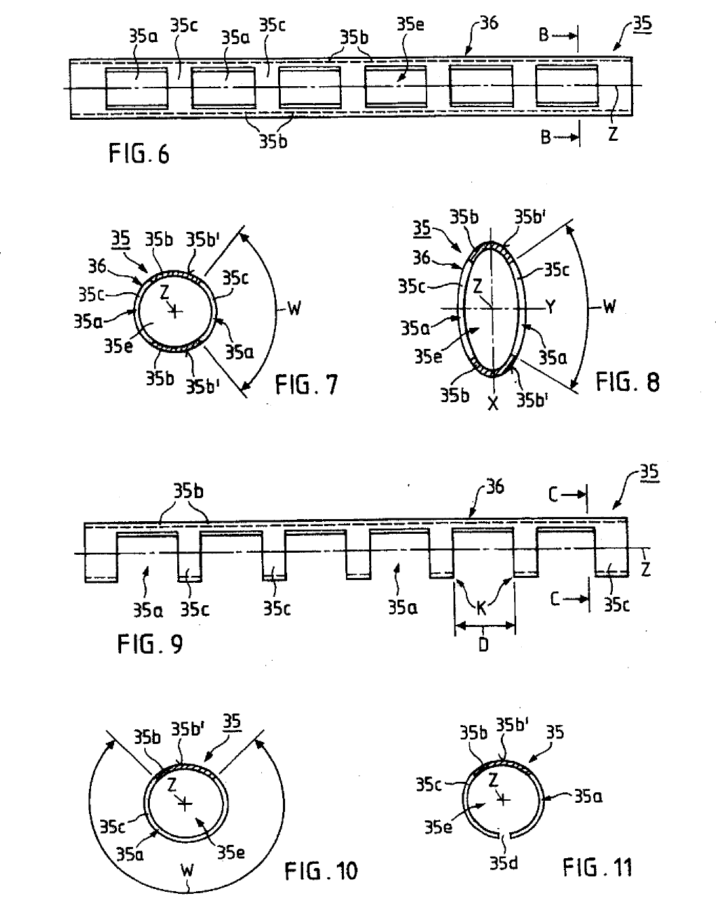

FIG. 6 shows a first embodiment of an implant made form a flexible tube 36, which includes two circumferentially disposed connecting parts 35b opposing each other and extending in direction of the longitudinal axis Z, as well as ring members 35c disposed in direction of the longitudinal axis Z and distanced from each other. Between each of the ring members 35c is an opening 35 which connects to the interior space 35e of implant 35. In the embodiment as shown, the openings 35a which connect to the interior space 35e are square shaped, however the openings 35a can have any shape, for example, oval, elliptical, square or trapeze shaped.

As this point, it is pointed out that the openings 35a, as well as the web shaped ring members disposed therebetween, as shown in FIG. 17, can be configured relative to the longitudinal axis Z, leaning either in one or the other direction, wherein each of the ring members 35c are disposed parallel and distanced from each other. It is possible that the ring members 35c, distanced by the openings 35a relative to the longitudinal axis Z, can be arranged alternately leaning in opposite direction. As shown in FIG. 18, the openings 35a can be arranged in series at one side of the tube 36, as well as the openings 35a′ in series at the opposite side of the tube 36, also in direction of the longitudinal section, and set off relative to each other.

The web shaped ring members 35c are preferably narrow and the openings 35a or recesses 35a oriented in axial direction, are formed relatively large such that when the implant 35 is inserted, as afore-described, the trabecular tissue 18 as well as each of the single small canals 21′. 22′ of canal system 20 are exposed in order to thus realize the natural transtrabecular flow of the aqueous humor (FIG. 5).

FIG. 7 shows an enlarged implant 35 formed with a circular ring shaped profile cross section according to section line B-B in FIG. 6, with the two connecting parts 35b disposed circumferentially opposite each other and oriented in the direction longitudinal axis Z. Furthermore, the openings 35a are shown between each of the connecting parts 35b, arranged in circumferential direction and connected to the interior space 35e. In this embodiment, the openings opposite each other between each of the web shaped ring members 35c, have an opening angle W delimited between 90° and 105°. The connecting parts 35b are provided with a circular surface oriented in the direction of the longitudinal axis Z, and configured for bearing at the interior wall 16′ of lumen 16 (FIG. 5).

FIG. 8 shows an enlarged view of a variant of the implant 35 according to the section line B-B of FIG. 6. Deviating from the embodiment as illustrated in FIG. 7, this implant 35 has an oval shaped profile cross section, preferably configured as a double symmetrical ring shaped oval that includes two symmetry axes X and Y oriented orthogonal to the longitudinal axis Z. The implant 35 constructed as a double symmetrical and ring shaped oval has two connecting parts 35b at the smaller circular ends and oriented in longitudinal direction having an arc shaped surface 35b′. FIG. 8 further shows the openings 35a at the larger circular shaped sides and opposite each other, connected to the interior space 35e, which, in the example of this embodiment, have an opening angle W each delimited between 90° and 105°.

FIG. 9 shows a further embodiment of the implant made from a flexible tube 36, which, deviating form the embodiment shown in FIG. 6, includes only a single continuous connecting part oriented in the direction of the longitudinal axis Z, as well as several ring members 35c that are distanced from each by openings 35a. In this embodiment, openings 35a each are a recess extending from one side of the connecting part 35b to the other side of connecting part 35b. The distance D between each of the web shaped ring members 35c, is selected such that in circular arc shaped condition of the implant 35 (not shown), the edges K of the web shaped ring members 35 are still at a distance from each other. The result is that the implant 35 inserted into Schlemm's canal 15 takes up a circumferentially oriented balanced position and a tilting of each of the ring member 35c is prevented. Each of the ring members 35c arranged in axial direction of the longitudinal axis Z and distanced from each other, are each provided at the exterior side, similar to the connecting part 35b, with an arc shaped surface 35b′. The ring members 35c shown in parallel distance from each other in FIG. 9 can be configured in axial direction either leaning in one direction, or alternately, leaning in opposite direction to each other.

FIG. 10 shows the implant 35 according to section line C-C in FIG. 9 in circular profile cross section with the connecting part 35b oriented in the direction of longitudinal axis Z having an arc shaped surface 35b′, as well as the recess 35a having a opening angle W of 280° to 290° and connected to the interior space 35c.

FIG. 11 illustrates a variant of implant 35 according to section line C-C according to FIG. 9 shown in profile cross section, where each of the ring members 35c of the circular shaped implant 35 is separated by an axially oriented slot 35d at the side opposite the connecting part 35b. Each of the ring members 35c, which for example can be detachably connected to a probe (inserting instrument) or similar, are bend upward relative to each other and returned to their original position due to their own spring elastic recoiling force.

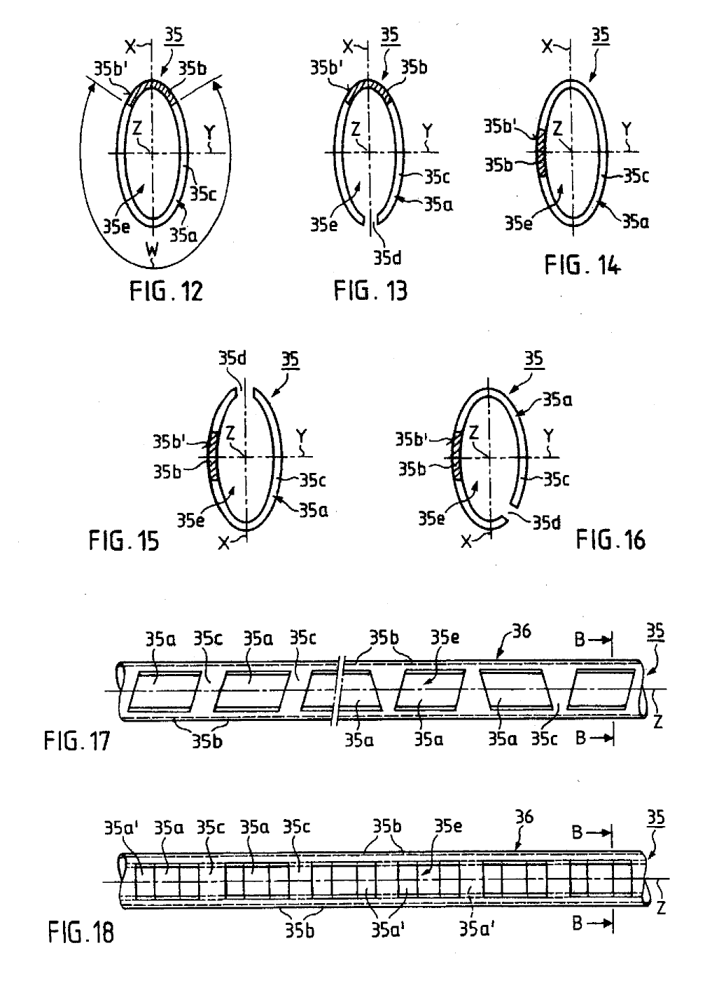

A further variant of the implant 35 according to the section line C-C in FIG. 9 is shown enlarged in FIG. 12. Deviating form the embodiment as shown in FIG. 10 or 11, the implant 35 according to FIG. 12, is configured with a circular oval shaped profile cross section, preferably as a double symmetrical ring shaped oval, with the longitudinal axis Z, as well as the two symmetry axes X and Y essentially arranged orthogonal thereto. In this variant, the connecting part 35b, having the arc shaped surface 35b′ and oriented in direction of the longitudinal axis Z, is arranged at the upper section of the oval. In another variant, not shown here, the connecting part 35b can be arranged however also opposite the lower arc shaped section of the oval.

FIG. 13 shows a variant of implant 35 according to FIG. 12, where the ring members 35c arranged at a distance from each other and oriented in direction of longitudinal axis Z, are each separated by a slot 35d at the end of the oval opposite the connecting part 35b. Each of the ring members 35c can thus be bend upward relative to each other, not shown here in detail, and returned to their original position by means of their own spring elastic recoiling force.

FIG. 14 shows a further embodiment of the implant 35 with an oval ring shaped profile cross section, preferably configured as a double symmetrical ring shaped oval, with the longitudinal axis Z, as well as the two symmetrical axes X and Y. In this implant 35, provided with an arc shaped surface 35b′, the connecting part 35b oriented in direction of the longitudinal axis Z, is arranged at the one side, or can be arranged at the other, opposite, arc shaped side.

FIG. 15 shows a variant of the implant 35 according to FIG. 14, where the ring members 35c arranged distanced to each other in the direction of the longitudinal axis Z, at the smaller arc shaped end of the oval, each are separated by a slot 35d. Slot 35d can however also be arranged at the other oppositely located arc shaped end of the oval.

FIG. 16 shows a further variant of implant 35, where, deviating from the variant as shown in FIG. 15, the ring members 35c arranged distanced to each other in the direction of the longitudinal axis Z, at the connecting part 35b, each are separated by a slot 35d circumferentially arranged at any location of the oval.

Relative to each implant as shown in FIGS. 13, 15 and 16, it is possible that each of the ring members 35c, separated by slot 35d, can be bend upward relative to the axis X, for example for the detachable connection with a probe (inserting instrument), and by its own spring elastic recoiling force, can return its original position.

The recesses 35a between each of the web shaped ring members 35c of implant 35 shown in FIGS. 12 to 16 configured as double symmetrical ring shaped oval, are each provided in circumferential direction of the oval, with an opening angle W of 280° to 290°.

The implant 35 made from an elongated tube shown and described in connection with each of the FIGS. 6 to 18, is made from a biocompatible flexible material, such as gold, nitinol or similar, or biocompatible flexible material such as polymeric material with thermal or mechanical shape memory. Thereby, the implant 35 which, for example, configured close to the circular shape like Schlemm's canal 15, is prior to insertion at a room temperature of about 18° to 22° C. mostly bend up straight and can be inserted into Schlemm's canal 15, where due to the body temperature of about 35° C. to 37°, and stays bearing against the inner wall 16′ in a shape corresponding to Schlemm's canal 15, can return to its shape. It is also possible, that the implant 35 prior to insertion, or while being inserted into Schlemm's canal 15, at a room temperature of about 18° to 22° C., is being somewhat compressed transverse to the longitudinal axis, not shown here in detail, and due to the body temperature of about 35° C. to 37° can return to the original circular or oval ring shaped shape.

Furthermore, there is the possibility that implant 35 made from flexible material, includes two connecting parts 35b (FIG. 7, 8) arranged axially opposite relative to each other or, is provided with just one connecting part 35b (FIGS. 10 to 16) oriented in axial direction. In a preferred embodiment it is possible that the circular ring shaped or oval ring shaped configured implant 35 made from an elongated tube 36, is provided with a heparin-coating.

For an insertion of the implant 35 into the lumen of Schlemm's canal 15, according to the afore-described canalo-plasty method, Schlemm's canal is being circularly carefully dilated and at the same time, or subsequently, injected, by means of a so-called microscrew, with a high molecular elasticoviscous agent. After dilation, the microcatheter is withdrawn and at the same time, Schlemm's canal 15 somewhat tensed toward the direction of the anterior chamber V, to thereby realize a stretching of the trabecular tissue 18, so that the implant 35 can be inserted. With the circular ring shaped flexible implant 35 (canaloplasty ring) according to the invention and shown as afore-described in detail, as well as in each of the FIGS. 6 to 18, on the one hand, a permanent dilation of the trabecular works is realized, and on the other hand, the lumen 16 of the circular Schlemm's canal 15 is circumferentially permanently held open and stabilized for the transtrabecular drainage of the aqueous humor.

1-21. (canceled)

22. An Implant for inserting into Schlemm's canal that has been exposed through an incision and the opening of a scleral flap of an eye, comprising:
an elongated flexible tube insertable into the lumen of Schlemm's canal, the tube comprising first and second axially extending connecting parts in diametrical disposition to each other and connected by web shaped ring members arranged at a distance from each other in axial direction at the tube, leaving openings between each of the oppositely arranged ring members that lead to an interior of the tube, wherein the recesses are each provided with a opening angle W oriented in circumferential direction.

23. The implant according to claim 22, wherein the opening angle (W) of a size between 90° to 105°.

24. The Implant according to claim 22, wherein the openings between the first and the second connecting part, as well as between each of the ring members are configured as circumferentially oriented recesses.

25. The implant according to claim 22, wherein the opening angle (W) is of the size between 280° to 290°.

26. The implant according to claim 22, wherein one of the connecting parts, is provided with a slot shaped axially continuous passage, whereby the two parts are expandable relative to each other.

27. The implant according to claim 22, wherein the web shaped ring members are each provided with a slot shaped passage and wherein the two connecting parts are expandable relative to each other.

28. The implant according to claim 22, wherein the elongated tube has a circular ring shaped profile cross section.

29. The implant according to claims 22, wherein the elongated tube has an oval ring shaped profile cross section.

30. The implant according to claim 22, wherein the elongated tube has a double symmetric oval ring shaped profile cross section.

31. The implant according to claim 27, wherein each of the ring members are separated by slot arranged anywhere at the circumference of the ring member.

32. The implant according to claim 22, wherein the openings connected to the interior space of the tube, are configured rectangular, square or trapezoidal and opposite each other.

33. The implant according to claims 22, wherein the openings are configured oval or elliptical and opposite each other.

34. The implant according to claim 32, wherein the openings provided at one side, as well as those at an opposite side of the tube are axially arranged set off from each other.

35. The implant according to claim 33, wherein the web shaped ring member are leaning in axial direction either in forward direction of rearward direction.

36. The implant according to claim 34, wherein each of the web shaped ring members are alternately and leaning opposite each other.

37. The implant according to claim 22, wherein the flexible tube has a length extending at least one to one half of the circumference of the exposed Schlemm's canal and provided at a distal end with a collar for bearing at the inner wall of the sclera when inserted into Schlemm's canal.

38. The implant according to claim 22, wherein the flexible tube has a length extending the entire circumference of the exposed Schlemm's canal.

39. The implant according to claim 22, wherein the tube is made of a biocompatible flexible material.

40. The implant according to claim 39, wherein the tube is from polymer material having thermal and mechanical shape memory

41. The implant according to claim 40, wherein the tube is flexible in arc shaped manner and ductile relative to a cross section where it is insertable into the lumen of the exposed circular Schlemm's canal, and due to a persons body temperature can be returned into the predetermined original shape.

42. The implant according to claim 39, wherein the tube is made of gold or nitinol.

43. The implant according to claim 39, wherein the tube is provided with a heparin-coating.
